(19) United States
(12) Patent Application Publication
(10) Pub. No.: US 2002/0128268 A1
(43) Pub. Date: Sep. 12, 2002

(54) Use of 2-hydrazino-1,3-thiazoles as antioxidants

(76) Inventor: Jakob Peter Ley, Holzminden (DE)

Correspondence Address: BAYER CORPORATION, PATENT DEPARTMENT, 100 BAYER ROAD, PITTSBURGH, PA, 15205, US

(21) Appl. No.: 10/001,589

(22) Filed: Oct. 19, 2001

(30) Foreign Application Priority Data
Oct. 24, 2000 (DE) 10052590.3

(51) Int. Cl. A61K 31/675; A61K 31/519; A61K 31/426

(52) U.S. Cl. 514/249; 514/260.1; 514/301; 514/363; 514/367; 514/370; 514/80

(57) ABSTRACT

2-Hydrazino-1,3-thiazoles, or their salts, can be used as effective constituents in cosmetic and dermatological preparations, and in preparations which are of use in nutrition or consumption, for protecting mammalian cells and tissues, and also the preparations, from the harmful effect of reactive oxygen species and free radicals.

### FIELD OF THE INVENTION

The invention relates to the use of 2-hydrazino-1,3-thiazoles as antioxidants or free radical-capturing agents, preferably as antioxidants or free radical-capturing agents in cosmetic or dermatological preparations and in preparations which are of use in nutrition or consumption. Furthermore, the invention relates to the use of these preparations for protecting mammalian cells and tissues from the aging-accelerating, harmful effects of free radicals and reactive oxygen compounds.

### BACKGROUND OF THE INVENTION

Reactive oxygen species and other reactive free radicals induce or intensify a number of diseases and types of aging damage in organisms, in particular mammals. Thus, for example, special aspects of the photoaging of the skin, damage to the retina in connection with age-associated macula degeneration, clouding of the lens in cataract, and special types of carcinogenesis and atheroscelerosis, are regarded as being causally related to the reaction of biologically important molecules, such as deoxyribonucleic acid or proteins, with free radicals.

Active compounds which, in physiological systems, in particular in mammals, support the natural mechanisms of defense against free radicals and reactive oxygen compounds, or, as protective compounds in cosmetics, pharmaceuticals or foodstuffs, protect the oxidation-sensitive constituents of the substances from autooxidation, are sought for cosmetic and dermatological preparations and for preparations which are of use in nutrition or consumption.

Antioxidants are substances which, in small concentrations as compared with the oxidizable substrate, significantly retard oxidation or completely prevent it. Many antioxidants, at the same time, function as free radical-capturing agents and/or as sequestering agents for heavy metal ions.

### SUMMARY OF THE INVENTION

It has been found that 2-hydrazino-1,3-thiazoles of the general formula can be used as antioxidants or free radical-capturing agents:

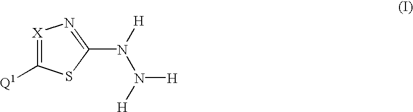

or their salts,

wherein

X represents a nitrogen atom or a substituted carbon atom C-Q², and

either

Q¹ and Q² represent, independently of each other, hydrogen atoms, optionally hydroxyl- or alkyloxy-substituted unbranched, branched or cyclic alkyl, alkenyl, 1-oxoalkyl or 1-oxoalkenyl groups having from 1 to 18 carbon atoms, optionally substituted aryl groups having from 6 to 15 carbon atoms, optionally substituted heterocyclyl groups having from 2 to 15 carbon atoms and at least one heteroatom selected from the group oxygen, nitrogen and sulfur, optionally substituted arylalkyl or aryl-1-oxoalkyl groups having from 7 to 16 carbon atoms, halogen atoms, nitro groups or —COOR⁴, —OR⁴, —NR⁴R⁵, —SO₂OR⁴, —SO₂NR⁴R⁵ or —PO(OR⁴)(OR⁵) groups,

or

Q¹ and Q² together represent a radical of the general formula (II)

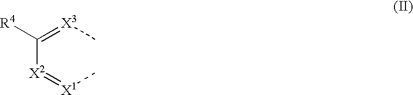

wherein

X¹, X² and X³ represent, independently of each other, either nitrogen atoms or carbon atoms having the radicals R¹, R² and R³, respectively, and

R¹, R² and R³ represent, independently of each other, hydrogen atoms, optionally hydroxyl- or alkyloxy-substituted unbranched, branched or cyclic alkyl, alkenyl, 1-oxoalkyl or 1-oxoalkenyl groups having from 1 to 18 carbon atoms, optionally substituted aryl groups having from 6 to 15 carbon atoms, optionally substituted heterocyclyl groups having from 2 to 15 carbon atoms and at least one heteroatom selected from the group oxygen, nitrogen and sulfur, optionally substituted arylalkyl or aryl-1-oxoalkyl groups of from 7 to 16 carbon atoms, halogen atoms, nitro groups or —COOR⁴, —OR⁴, —NR⁴R⁵, —SO₂OR⁴, —SO₂NR⁴R⁵ or —PO(OR⁴)(OR⁵) groups, and

R⁴ and R⁵ represent, independently of each other, hydrogen atoms, optionally hydroxyl- or alkyloxy-substituted unbranched, branched or cyclic alkyl, alkenyl, 1-oxoalkyl or 1-oxoalkenyl groups having from 1 to 18 carbon atoms, optionally substituted aryl groups having from 6 to 15 carbon atoms, or optionally substituted arylalkyl or aryl-1-oxoalkyl groups of from 7 to 16 carbon atoms.

The 2-hydrazino-1,3-heteroazoles according to the present invention can also be present in the form of their tautomers.

An unbranched, branched or cyclic alkyl group can contain from 1 to 18, preferably from 1 to 8, more preferably from 1 to 6, carbon atoms. Examples which may be mentioned are: methyl, ethyl, 1-propyl, 2-propyl-, 1-butyl, 2-butyl, tert-butyl, 2-methyl, 2-methylprop-1-yl, cyclopropyl, cyclopropylmethyl, 2,2-dimethylcyclopropyl, cyclobutyl, cyclopentyl, cyclohexyl and different positional isomers of methylpentyl.

An unbranched, branched or cyclic alkenyl group can contain from 2 to 18, preferably from 2 to 8, more preferably from 2 to 6 carbon atoms. Examples which may be mentioned are: ethenyl, 1- or 2-propenyl, 1-, 2- or 3-butenyl, 2-methyl-I-propenyl, 2-methyl-2-propenyl, 1,3-butadienyl, 1,3-pentadienyl, 1,4-pentenyl, 2,4-pentenyl, and the respective different straight-chain, cyclic or branched isomers of the pentenyl and hexenyl radicals. Ethenyl, 1- or 2-propenyl, 1-, 2- or 3-butenyl, 2-methyl-1-propenyl, 2-methyl-2-propenyl, 3-methyl-1-pentenyl, 3-methyl-2-pentenyl, 3-Methyl-3-pentenyl, cyclopentenyl, cyclopentadienyl, cyclohexadienyl and cyclohexenyl are more preferred.

An unbranched, branched or cyclic 1-oxo-alkyl group can contain from 1 to 18, preferably from 1 to 8, and more preferably from 1 to 5 carbon atoms. Examples which may be mentioned are: formyl, acetyl, propionyl, 2-methylpropionyl, butanoyl, 2-methylbutanoyl, 3-methylbutanoyl, 2,2-dimethylpropionyl and cyclopropylcarboxyl.

An unbranched, branched or cyclic 1-oxo-alkenyl group can contain from 3 to 18, preferably from 3 to 8, and more preferably from 3 to 5 carbon atoms. Examples which may be mentioned are: prop-2-enoyl, 2-methylprop-2-enoyl, 2-ethyl-prop-2-enoyl, E- or Z- 2-butenoyl, 3-butenoyl, E- or Z- 2-methylbut-2-enoyl, E- or Z- 3-methylbut-2-enoyl, Z- or E- 2-pentenoyl, Z- or E- 3-pentenoyl.

Aryl groups having from 6 to 15 carbon atoms, preferably from 6 to 10 carbon atoms, can, for example, be phenyl and naphthyl.

A heterocyclyl group having from 2 to 15 carbon atoms and at least one atom from the group oxygen, sulfur and nitrogen in the ring generally contain from 1 to 3, preferably 1 or 2, five- or six-membered rings. The heterocyclyl group preferably contains from 1 to 4, preferably 1 or 2, heteroatoms. Furan, pyrrole, thiophene, indole, isoindole, benzofuran, Isobenzofuran, benzothiophene, isobenzothiophene, pyrazole, imidazole, 1,3- or 1,2-oxazole, 1,3- or 1,2-thiazole, 1,3- or 1,2-benzimidazole, 1,3- or 1,2-benzoxazole, 1,3- or 1,2-benzothiazole, pyridine, pyrimidine, pyrazine, 1,2-, 1,3- or 1,4-oxazine, 1,2-, 1,3- or 1,4-thiazine, quinoline, isoquinoline, benzo-1,2-, -1,3- or -1,4-diazine or their partially or completely saturated derivatives, e.g. tetrahydrofuran, 1,3-dioxolane, pyrrolidine, pyrroline, 1,3- or 1,4-dioxane, piperidine, tetrahydro-2H-pyran, piperazine, oxirane or aziridine are preferred. Furan, pyrrole, indole, imidazole, 1,3-thiazole, 1,3-benzothiazole, pyridine, pyrimidine, quinoline, isoquinoline or their partially or completely saturated derivatives, e.g. tetrahydrofuran, 1,3-dioxolane, pyrrolidine, 1,3- or 1,4-dioxane, piperidine or tetrahydro-2H-pyran are more preferred.

An arylalkyl group can contain from 6 to 15 carbon atoms, preferably from 7 to 8 carbon atoms, and can, for example, be: benzyl, or 2- or 1-phenylethyl.

An aryl-1-oxo-alkyl group can contain 6 to 15 carbon atoms, preferably from 7 to 8 carbon atoms, and can, for example, be: benzoyl, or phenylacetyl.

Substituents of the aryl, arylalkyl, aryl-1-oxoalkyl and heterocyclyl groups can, for example, be: hydrogen atoms, or alkyl, hydroxyl, alkyloxy, thio-, alkylthio, amino, alkylamino, dialkylamino, nitro, iodine, bromine, fluorine, chlorine, azido, thiocyanato, isothiocyanato, cyanato, isocyanato, nitrile, isonitrile, phosphate, alkyl phosphate, dialkyl phosphate, sulfonic acid, alkylsulfonate, sulfonamide, dialkylsulfonamide or alkylsulfonamide radicals. Hydrogen atoms, or alkyl, hydroxyl, alkyloxy, amino, dialkylamino, bromine, fluorine, chlorine, nitrile, sulfonic acid, sulfonamide or alkylsulfonate radicals are more preferred.

Preference is given to using the 2-hydrazino-1,3-thiazoles of the general formula (I)

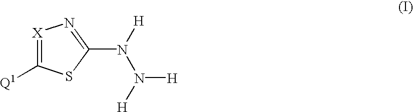

or their salts,

wherein

X represents a nitrogen atom or a Q²-substituted carbon atom C-Q², and

either

Q¹ and Q² represent, independently of each other, hydrogen atoms, methyl, trifluoromethyl, ethyl, tert-butyl, allyl, 3-methylbut-2-en-1-yl or acetyl groups, chlorine or fluorine atoms, nitro groups, —COOR⁴, —OR⁴, —NR⁴R⁵, —SO₂OR⁴, —SO₂NR⁴R⁵ or —PO(OR⁴)(OR⁵) groups or phenyl, pyridyl, pyrazinyl, phenylmethyl or benzoyl groups which are optionally substituted on the aromatic moiety by alkyl, hydroxyl, alkyloxy, amino, dialkylamino, bromine, fluorine, chlorine, nitrile, sulfonic acid, sulfonamide or alkylsulfonate radicals, or

Q¹ and Q² together represent a radical of the general formula (II)

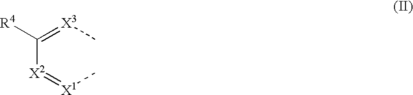

where

X¹, X² and X³ represent carbon atoms having the radicals R¹, R² and R³, respectively, and

R¹, R² and R³ represent, independently of each other, hydrogen atoms, methyl, trifluoromethyl, ethyl, tert-butyl, allyl, 3-methylbut-2-en-1-yl- or acetyl groups, phenyl, pyridyl, pyrazinyl, phenylmethyl, 4-methylphenylmethyl, 4-methoxyphenylmethyl or benzoyl groups, 5 chlorine or fluorine atoms, nitro groups, or —COOR⁴, —OR⁴, —NR⁴R⁵, —SO₂OR⁴, —SO₂NR⁴R⁵ or —PO(OR⁴)(OR⁵) groups and

R⁴ and R⁵ represent, independently of each other, hydrogen atoms, methyl, trifluoromethyl, ethyl, tert-butyl, allyl, 3-methylbut-2-en-1-yl, acetyl, propionyl or pivaloyl groups, or phenyl, pyridyl, pyrazinyl, phenylmethyl, 4-methylphenylmethyl, 4-methoxyphenylmethyl or benzoyl groups, as antioxidants or free radical-capturing agents.

More preference is given to the use of 2-hydrazino-1,3-benzothiazole 5,6-dimethoxy-2-hydrazino-1,3-benzothiazole 6-methoxy-2-hydrazino-1,3-benzothiazole 2-hydrazino-i,3-benzothiazole-5-sulfonic acid 2-hydrazino-1,3-benzothiazole-6-sulfonic acid 6-tert-butyl-2-hydrazino-1,3-benzothiazole 6-methyl-2-hydrazino-1,3-benzothiazole 2-hydrazino-thiazolo-[5,4-b]pyridine 2-hydrazino-5-(4-fluorophenyl)-1,3,4-thiadiazole 2-hydrazino-4-phenyl-1,3-thiazole 2-hydrazino-4-methyl-1,3-thiazole-hydrochloride as antioxidants or free-radical capturing agents.

Surprisingly, it has now been found that the 2-hydrazino-1,3-thiazoles according to the present invention are very good free radical-capturing agents and strong antioxidants. They are preferably suitable for use as free radical-capturing agents. In particular, the 2-hydrazino-1,3-thiazoles according to the present invention are able to suppress the harmful effects of free radicals and/or oxidative processes, which are, for example, induced by UV light, on and/or in the human skin and to support the natural antioxidative processes. They can, therefore, be used as active compounds in cosmetic or dermatological skin clearing compositions. In addition, the 2-hydrazino-1,3-thiazoles according to the present invention constitute very good antioxidants for highly unsaturated lipids, such as squalene, lycopene, carotenes, docosahexaenoic acid, eicosapentaenoic acid, α- or γ-linolenic acid or linoleic acid, and also for fatty oils containing (poly) unsaturated fatty acids, such as soyabean oil, linseed oil, thistle oil, borage seed oil, night candle oil, fish oil olive oil and sunflower oil. They can, therefore, be used as antioxidants in preparations which are of use in nutrition or consumption and which contain such lipids. In particular, the 2-hydrazino-1,3-thiazoles according to the present invention are also outstandingly suitable for stabilizing the pure lipids or fatty oils or mixtures thereof.

The 2-hydrazino-1,3-thiazoles according to the present invention can be used against the harmful effect of free radicals and reactive oxygen species, preferably in pharmaceutical, cosmetic or dermatological preparations, preferably for protecting mammalian cells and tissues, particularly the human skin, and in foodstuffs. It is naturally also possible to employ the 2-hydrazino-1,3-thiazoles according to the present invention, and/or the preparations comprising such 2-hydrazino-1,3-thiazoles, in an analogous manner in other areas of use.

The invention, therefore, also relates to cosmetic and dermatological preparations, and preparations which are of use in nutrition or consumption, comprising the 2-hydrazino-1,3-thiazoles of the general formula (I)

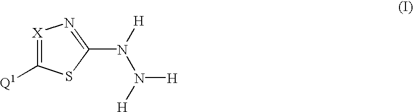

or their salts,

wherein

X represents a nitrogen atom or a substituted carbon atom C-Q², and

either

Q¹ and Q² represent, independently of each other, hydrogen atoms, methyl, trifluoromethyl, ethyl, tert-butyl, allyl, 3-methylbut-2-en-1-yl or acetyl groups, chlorine or fluorine atoms nitro groups, —COOR⁴, —OR⁴, —NR⁴R⁵, —SO₂OR⁴, —SO₂NR⁴R⁵ or —PO(OR⁴)(OR⁵) groups or phenyl, pyridyl, pyrazinyl, phenylmethyl or benzoyl groups which are optionally substituted on the aromatic moiety by alkyl, hydroxyl, alkyloxy, amino, dialkylamino, bromine, fluorine, chlorine, nitrile, sulfonic acid, sulfonamide or alkylsulfonate radicals, or

Q¹ and Q² together represent a radical of the general formula (II)

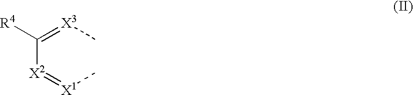

where

X¹, X² and X³ represent carbon atoms having the radicals R¹, R² and R³, respectively, and

R¹, R² and R³ represent, independently of each other, hydrogen atoms, methyl, trifluoromethyl, ethyl, tert-butyl, allyl, 3-methylbut-2-en-1-yl- or acetyl groups, phenyl, pyridyl, pyrazinyl, phenylmethyl, 4-methylphenylmethyl, 4-methoxyphenylmethyl or benzoyl groups, chlorine or fluorine atoms, nitro groups, or —COOR⁴, —OR⁴, —NR⁴R⁵, —SO₂OR⁴, —SO₂NR⁴R⁵ or —PO(OR⁴)(OR⁵) groups, and

R⁴ and R⁵ represent, independently of each other, hydrogen atoms, methyl, trifluoromethyl, ethyl, tert-butyl, allyl, 3-methylbut-2-en-1-yl, acetyl, propionyl or pivaloyl groups, or phenyl, pyridyl, pyrazinyl, phenyl methyl, 4-methylphenylmethyl, 4-methoxyphenylmethyl or benzoyl groups.

In physiological systems, the cosmetic and dermatological preparations according to the present invention, and the preparations according to the present invention which are of use in nutrition or consumption, support the natural mechanisms of defense against free radicals and reactive oxygen compounds in physiological systems and, in cosmetics, pharmaceuticals, foodstuffs of consumables, protect their oxidation-susceptible constituents from autooxidation or photooxidation.

The 2-hydrazino-1,3-thiazoles, some of which are known, can be prepared, for example, from the 2-amino-1,3-thiazoles using the method described in Organic Preparations and Procedures Int. 1974, 6(4), 179-182. The 2-amino-1,3-thiazoles can be prepared, for example, from the corresponding anilines or heterocyclic amines by reaction with inorganic thiocyanate salts and subsequent oxidative ring closure of the N-arylthiourea (J. Indian Chem. Soc. 1989, 66, 39-41).

The 2-hydrazino-1,3-benzothiazoles according to the present invention can be prepared, for example, from an optionally substituted aniline, by reaction with potassium, sodium or ammonium thiocyanate, followed by chlorine-, bromine- or iodine-mediated oxidative ring closure and then, finally, by reaction with hydrazine or hydrazine hydrate.

The preparation can be explained by the following formula scheme, which uses 2-hydrazino-6-tert-butyl-1,3-benzothiazole as an example:

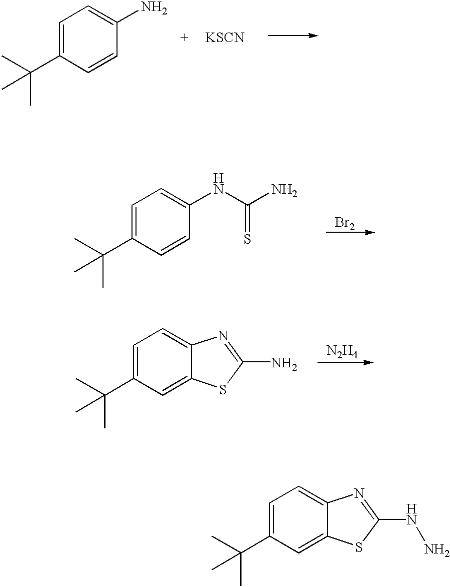

The preparations according to the present invention, which comprise the 2-hydrazino-1,3-thiazoles, are produced by means of customary methods which are known per se such that one or more of the 2-hydrazino-1,3-heteroazoles of the general formula I according to the present invention, or their salts, is/are incorporated into the formulations, which have a customary composition and which can be used for treating, protecting, caring for and cleaning the skin or hair, as make-up products and as foodstuffs or consumables.

The preparations according to the present invention comprise 2-hydrazino-1,3-thiazoles in an effective quantity and other constituents where appropriate. They comprise from 0.0001% by weight to 30%, by weight, preferably from 0.001 to 20% by weight, in particular, however, from 0.001% by weight to 5% by weight, of the 2-hydrazino-1,3-thiazoles according to the present invention, based on the total weight of the formulation, and can be present, in this context, as “water in oil”, “oil in water”, “water in oil in water” or “oil in water in oil” emulsions, as microemulsions, as gels, or as solutions, for example, in oils, alcohols or silicone oils. The preparations can comprise other customary auxiliary substances and additives in quantities from 5 to 99.9999% by weight, preferably from 10 to 80% by weight, based on the total weight of the formulation. In addition, the formulations can comprise water in a quantity up to 99.9999% by weight, preferably from 5 to 80% by weight, based on the total weight of the formulation.

In another embodiment, the 2-hydrazino-1,3-thiazoles of the general formula I can, for producing the preparations according to the present invention, also be incorporated beforehand into liposomes, for example, prepared from phosphatidylcholine, into microspheres, into nanospheres or else into capsules composed of a suitable matrix, for example, composed of natural or synthetic waxes, for example beeswax, carnauba wax, silicone wax or stearyl alcohol, eicosanol, cetyl alcohol, stearin or paraffin wax, or composed of gelatin. In another embodiment, 2-hydrazino-1,3-thiazoles of the general formula I are complexed beforehand with sequestering agents, for example with cyclodextrins or cyclodextrin derivatives, preferably methylcyclodextrin.

The cosmetic and dermatological preparations according to the present invention can comprise cosmetic auxiliary substances and additives as are customarily used in such preparations, for example sunscreen agents (e.g. organic or inorganic light-filtering substances, preferably micropigments), preservative, bactericides, fungicides, virucides, coolants, plant extracts, antiinflammatory active compounds, wound healing-accelerating substances (e.g. chitin or chitosan and its derivatives), film-forming substances (e.g. polyvinylpyrrolidones or chitosan or its derivatives), customary antioxidants, vitamins, (e.g. Vitamin C and derivatives, tocopherols and derivatives, Vitamin A and derivatives) 2-hydroxycarboxylic acids (e.g. citric acid, malic acid, and L-, D- or DL-lactic acid), skin clearing agents, (e.g. kojic acid, hydroquinone or arbutin), skin coloring agents (e.g. walnut extracts or dihydroxyacetone), perfumes, substances for preventing foaming, dyes, pigments which have a coloring effect, thickeners, surface-active substances, emulsifiers, emollient, moistening and/or moisture-retaining substances (e.g. glycerol or urea), fats, oils, unsaturated fatty acids or their derivatives (e.g. linoleic acid, -linolenic acid, γ-linolenic acid or arachidonic acid and their respective natural or synthetic esters), waxes or other customary constituents of a cosmetic or dermatological formulation such as alcohols, polyols, polymers, foam stabilizers, electrolytes, organic solvents, silicone derivatives and chelating agents (e.g. ethylenediaminetetraacetic acid and derivatives.

The skilled person can readily determine the quantities of cosmetic or dermatological auxiliary substances and additives, and perfume, which are to be employed in each case, in dependence on the nature of the respective product, by means of simple trial and error.

For use, the cosmetic or dermatological preparations according to the present invention, comprising 2-hydrazino-1,3-thiazoles of the general formula 1, are applied to the skin and/or the hair in the manner which is customary for cosmetics and in adequate quantity.

It is preferably possible for the cosmetic or dermatological preparations according to the present invention, comprising 2-hydrazino-1,3-thiazoles of the general formula I or their salts, also to contain active compounds for skin clarification. In particular, the topical cosmetic compositions according to the present invention can also comprise benzaldoximes containing at least one aromatic hydroxyl or alkoxy group, cojic acid, cojic acid derivatives, ascorbic acid, ascorbic acid derivatives, hydroquinone, hydroquinone derivatives, sulfur-containing molecules (e.g. glutathione or cysteine) or other synthetic or natural active compounds for skin clarification, with it also being possible to use the latter in the form of an extract from plants (e.g. tocopherols and derivatives, arbutin (e.g. from bearberry extract), aloesin (e.g. from aloe extract), grapefruit extract and rice extract).

In the cosmetic or dermatological preparations according to the present invention, the quantity of the active compounds for skin clarification (one or more compounds), which were mentioned above by way, of example, can be from 0.001 to 30% by weight, preferably from 0.001 to 20% by weight, more preferably from 0.001 to 5% by weight, based on the total weight of the preparation.

Preference is given to those cosmetic or dermatological preparations which are simultaneously present in the form of a sunscreen composition. In addition to an effective quantity of the 2-hydrazino-1,2-thiazoles of the general formula 1, these preparations also comprise sunscreen substances, preferably organic or inorganic light-filtering substances, in particular micropigments. However, the skin clarification compositions according to the present invention can also comprise UVA-and/or UVB-filtering substances, with it being possible for the total quantity of filtering substances to be from 0.1 to 30% by weight, preferably from 0.5 to 10% by weight, based on the total weight of the preparations, resulting in sunscreen compositions for the skin and the hair. Examples of UV-filtering substances are 3-benzylidenecamphor derivatives (e.g.. 3-(4-methylbenzylidene)-dI-camphor), aminobenzoic acid derivatives (e.g. 2-ethylhexyl 4-(N,N-dimethylamino)benzoate or menthylanthranilate), 4-methoxycinnamates (e.g. 2-ethylhexyl p-methoxycinnamate or isoamyl p-methoxycinnamate), benzophenones (e.g. 2-hydroxy-4-methoxybenzophenone), monosulfonated or polysulfonated UV filters (e.g. 2-phenylbenzimidazole-5-sulfonic acid, Sulisobenzone or 1,4-bis(benzimidazolyl)-benzene-4,4′,6,6′-tetrasulfonic acid or 3,3′-(1,4-phenylenedimethylidene)-bis-(7,7-dimethyl-2-oxo-bicyclo-[2,2,1]heptane-1-methanesulfonic acid) and their salts], salicylates (e.g. 2-ethylhexyl salicylate or homomenthyl salicylate), triazines {e.g.. 2,4-bis-[4-(2-ethylhexyloxy)-2-hydroxyphenyl]-6-(4-methoxyphenyl)-1,3,5-triazine, and bis(2-ethylhexyl) 4,4′-([6-([(1,1-dimethylethyl)-aminocarbonyl]-phenylamino)-1,3,5-triazin-2,4-diyl]diimino)bisbenzoate}, 2-cyanopropenoic acid derivatives (e.g. 2-ethylhexyl 2-cyano-3,3-diphenyl-2-propenoate), dibenzoyl derivatives (e.g. 4-tert-butyl-4′-methoxydibenzoylmethane), polymer-bound UV filters (e.g. polymers of N-[2-(or 4)-(2-oxo-3-bornylidene)methyl]benzylacrylamide) or pigments (e.g. titanium dioxides, zirconium dioxides, iron oxides, silicon dioxides, manganese oxides, aluminum oxides, cerium oxides or zinc oxides).

In another preferred embodiment of the invention, other antioxidants or free radical-capturing agents are also present in the preparations according to the present invention, in addition to the 2-hydrazino-1,3-thiazoles according to the present invention. In particular, it is possible to use any of the antioxidants, which are suitable or customary for the applications according to the present invention as the other antioxidants. The antioxidants are advantageously selected from the group of amino acids (e.g. glycine, histidine, 3,4-dihydroxyphenylalanine, tyrosine and tryptophan) and their derivatives, imidazoles (e.g. urocanic acid) and their derivatives, peptides (D,L-carnosine, D-carnosine, L-carnosine and anserine) and their derivatives, carotenoids, carotenes (e.g. β-carotene, β-carotene and lycopene) and their derivatives, chlorogenic acid and its derivatives, lipoic acid and its derivatives, aurothioglucose, propylthiouracil and other thiols (e.g. thioredoxin, glutathione, cysteine, cystine, cystamine and their glycosyl and N-acyl derivatives or their alkyl esters) and also their salts, dilauryl thiodipropionate, distearyl thiodipropionate, thiodipropionic acid and their derivatives, and also phenolic amides of phenolic benzylamines (e.g. homovanillic, 3,4-dihydroxyphenylacetic, ferulic, sinapic, caffeic, dihydroferulic, dihydrocaffeic, vanillomandelic or 3,4-dihydroxymandelic amides of 3,4-dihydroxybenzyl-, 2,3,4-trihydroxybenzyl- or 3,4,5-trihydroxybenzylamine), and, in addition, (metal)chelators (e.g. 2-hydroxy fatty acids, phytic acid and lactoferrin), humic acid, bile acids, bile extracts, bilirubin, biliverdin, folic acid and its derivatives, ubiquinone and ubiquinol and their derivatives, Vitamin C and its derivatives (e.g., ascorbyl palmitate, magnesium ascorbyl phosphate and ascorbyl acetate), tocopherols and derivatives (e.g. vitamin E acetate), vitamin A and derivatives (e.g. vitamin A palmitate), rutinic acid and its derivatives, flavonoids (e.g. quercetin and -glucosylrutin) and their derivatives, phenolic acids (e.g. gallic acid and ferulic acid) and their derivatives (e.g. propylgallate, ethylgallate and octylgallate), furfurylidene glucitol, dibutylhydroxytoluene, butylhydroxyanisole, uric acid and its derivatives, mannose and its derivatives, zinc and its derivatives (e.g. ZnO and ZnSO₄), selenium and its derivatives (e.g. selenomethionine), stilbenes and their derivatives (e.g. stilbene oxide and resveratrol) and the derivatives of these said active compounds which are suitable in accordance with the present invention.

The quantity of the other antioxidants (one or more compounds) mentioned above, in which the antioxidants are not identical to the 2-hydrazino-1,3-thiazoles according to the present invention, in the preparations according to the present invention is preferably from 0.0001 to 30% by weight, more preferably from 0.01 to 10% by weight, and more preferably from 0.01 to 5% by weight, based on the total weight of the preparations.

Apart from the 2-hydrazino-1,3-thiazoles according to the present invention, it is naturally also possible to use several additional antioxidants.

The lipid phase in the cosmetic or dermatological preparations according to the present invention, comprising 2-hydrazino-1,3-thiazoles of the general formula I or their salts, can advantageously be selected from the following substance groups: mineral oils (advantageously paraffin oil), mineral waxes, carbohydrates (advantageously squalane or squalene), synthetic or semisynthetic triglyceride oils (e.g. triglycerides of capric or caprylic acid), natural oils (e.g. castor oil, olive oil, sunflower oil, soybean oil, groundnut oil, rapeseed oil, almond oil, palm oil, coconut oil, palm kernel oil, borage seed oil and similar), natural ester oils, (e.g. jojoba oil, synthetic ester oils) preferably esters of saturated and/or unsaturated linear and/or branched alkanecarboxylic acids having from 3 to 30 C atoms with saturated and/or unsaturated, linear and/or branched alcohols having from 3 to 30 C atoms, and esters of aromatic carboxylic acids with saturated and/or unsaturated, linear and/or branched alcohols having from 3 to 30 C atoms, selected, in particular, from the group isopropyl myristate, isopropyl stearate, isopropyl palmitate, ispropyl oleate, n-butyl stearate, n-hexyl laurate, n-decyl laurate, isooctyl stearate, isononyl stearate, isononyl isononanoate, 2-ethylhexyl palmitate, 2-ethylhexyl laureate, 2-hexyldecyl stearate, 2-octyldecyl palmitate, oleyl oleate, oleyl erucate, erucyl oleate, erucyl erucate and synthetic or natural mixtures of such esters, fats, waxes and other natural and synthetic fatty compounds, preferably esters of fatty acids with alcohols of low C number (e.g. with isopropanol, propylene glycol or glycerol) or esters of fatty alcohols with alkanoic acids of low C number or with fatty acids, alkyl benzoates (e.g. mixtures of n-dodecyl, n-tridecyl, n-tetradecyl and n-pentadecyl benzoate) and also cyclic or linear silicone oils (such as dimethylpolysiloxanes, diethylpolysiloxanes, diphenylpolysiloxanes, and also mixed forms thereof).

Where appropriate, the aqueous phase of the cosmetic or dermatological preparations according to the present invention, comprising 2-hydrazino-1,3-thiazoles of the general formula I or their salts, advantageously contains alcohols, diols or polyols of low C number, and also their ethers, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, and, in addition, alcohols of low C number, e.g. ethanol, isopropanol, 1,2-propanediol and glycerol, and also, in particular one or more thickeners which can advantageously be selected from the group, silicon dioxide, aluminum silicates, polysaccharides or their derivatives, e.g. hyaluronic acid, xanthan gum, hydroxypropyl methyl cellulose, particularly advantageously from the group of the polyacrylates, preferably a polyacrylate from the group of what are termed the carbopols, in each case individually or in combination, or from the group of the polyurethanes.

More preference is given to using the preparations according to the present invention for protecting mammalian tissues and cells from oxidative stress. The cosmetic or dermatological preparations according to the present invention are used, in particular, for protecting human skin, hair and/or nails from oxidative stress and the harmful effect of free radicals.

The present invention also encompasses a process for protecting cosmetic and dermatological preparations, and also preparations which are of use in nutrition or consumption, from oxidation or photooxidation, with it being possible for these preparations to be, for example, preparations for treating, protecting or caring for the skin, the nails or the hair, or else foodstuffs and consumables whose constituents give rise to stability problems because of oxidation or photooxidation during storage, characterized in that preparations according to the present invention comprise an effective content of 2-hydrazino-1,3-thiazoles according to the present invention.

### EXAMPLES

### Example 1

“Oil in water” emulsion

TABLE 1

| | Name of | | Content |
|---|---|---|---|
| | raw material | | in % by |
| Part | (manufacturer) | Chemical designation | weight |
| A | Arlatone 983 S ® | Ether of polyethylene glycol with | 1.2 |
| | (ICl) | glyceryl monostearate | |
| | Brij 76 ® (ICl) | 3,6,9,12,15,18,21,24,27,30,33,36- | 1.2 |
| | | Decaoxaoctatetracontan-1-ol | |
| | Cutina MD ® | Glyceryl monostearate | 3.5 |
| | (Henkel) | | |
| | Baysilone oil | Polydimethylsiloxane | 0.8 |
| | M10 ® (GE Bayer) | | |
| | Eutanol G ® | Octyldodecanol | 3.0 |
| | (Henkel) | | |
| | Paraffin oil 65 cp | Mineral oil | 8.0 |
| | (Henry Lamotte) | | |
| B | Water, dist. | | 49.6 |
| | Phenopip ® (Nipa | 2-Phenoxyethanol and methyl 4- | 0.5 |
| | Laboratories) | hydroxybenzoate and ethyl 4- | |
| | | hydroxybenzate and propyl 4- | |
| | | hydroxybenzoate and butyl 4- | |
| | | hydroxybenzoate | |
| | Trilon BD ® | Disodium EDTA | 0.1 |
| | (BASF) | | |
| | 1,2-Propylene | | 2.0 |
| | glycol | | |
| | Glycerol, 99% | | 3.0 |
| | 2-Hydrazino-1,3- | | 0.2 |
| | benzothiazol-5- | | |
| | sulfonic acid | | |
| C | Water, dist. | | 25.0 |
| | Carbopol 2050 ® | Crosslinked Acrylic acid/C₁₀-C₃₀- | 0.4 |
| | (B. F. Goodrich) | alkyl acrylate polymer | |
| | Aqueous sodium | | 1.2 |
| | hydroxide solution, | | |
| | 10% | | |
| D | Perfume oil | | 0.3 |

Part A was mixed and heated to 80° C. Part B was mixed and heated to 90° C.and added to Part A while stirring. For part C, carbopol was carefully dispersed in water and neutralized (pH 6.9) with sodium hydroxide solution. Part C was then added, at 60° C., to the mixture consisting of parts A and B. Part D was then added at room temperature to the mixture consisting of parts A, B and C.

### Example 2

“Water in oil” sunscreen emulsion giving UVA/B broad-band protection

TABLE 2

| | | | Content |
|---|---|---|---|
| | Name of raw material | | in % by |
| Part | (manufacturer) | Chemical designation | weight |
| A | Dehymuls PGPH ® | Polyglycerol-2 | 3.0 |
| | (Henkel) | dipolyhydroxystearate | |
| | Monomuls 90-O 18 ® | Glyceryl oleate | 1.0 |
| | (Henkel) | | |
| | Permulgin 2550 ® | Beeswax | 1.0 |
| | (Koster Keunen | | |
| | Holland) | | |
| | Myritol 318 ® (Henkel) | Caprylic/capronic acid | 6.0 |
| | | triglycerides | |
| | Witconol TN ® (Witco) | C₁₂-C₁₅-alkyl benzoate | 6.0 |
| | Cetiol SN ® (Henkel) | Cetyl and stearyl | 5.0 |
| | | isononanoate | |
| | Copherol 1250 ® | Tocopherol acetate | 1.0 |
| | (Henkel) | | |
| | Solbrol P ® (Bayer) | Propyl 4-hydroxybenzoate | 0.1 |
| | Neo Heliopan ® AV | 2-Ethylhexyl p- | 4.0 |
| | (Haarmann & Reimer) | methoxycinnamate | |
| | Neo Heliopan ® E 1000 | Isoamyl p-methoxycinnamate | 4.0 |
| | (Haarmann & Reimer) | | |
| | Neo Heliopan ® MBC | 3-(4-Methylbenzylidene)-dl- | 2.0 |
| | (Haarmann & Reimer) | camphor | |
| | Neo Heliopan ® OS | 2-Ethylhexyl salicylate | 3.0 |
| | (Haarmann & Reimer) | | |
| | Octyltriazone | | 1.0 |
| | Zinc oxide, neutral | | 7.0 |
| | (Haarmann & Reimer) | | |
| B | Water, dist. | | 39.8 |
| | Trilon BD ® (BASF) | Disodium EDTA | 0.1 |
| | Phenoxyethanol | | 0.7 |
| | Solbrol M (Bayer) | Methyl 4-hydroxybenzoate | 0.2 |
| | Glycerol 99% | | 4.0 |
| | Neo Heliopan ® Hydro | 2-Phenylbenzimidazole-5- | 10.0 |
| | (Haarmann & Reimer), | sulfonic acid | |
| | 15% as sodium salt | | |
| | Benzophenone-4 | | 0.5 |
| | 2-Hydrazino-5-(4- | | 0.2 |
| | fluorophenyl)-1,3,4- | | |
| | thiadiazole | | |
| C | Perfume oil | | 0.3 |
| | Bisabol | | 0.1 |

For part A, all the substances apart from the zinc oxide were heated to 85° C. and the zinc oxide was then carefully dispersed in the mixture. The components of part D were mixed, heated to 85° C. and added to part A while stirring. Part C was added to the mixture consisting of parts A and B and the mixture was subsequently homogenized using a dispersing implement.

### Example 3

“Oil in water” sunscreen emulsion giving UVA/B broad-band protection

TABLE 3

| | | | Content |
|---|---|---|---|
| | Name of raw material | | in % by |
| Part | (manufacturer) | Chemical designation | weight |
| A | Arlacel 165 ® (ICl) | Glyceryl stearate and | 3.0 |
| | | polyethyleneglycol 100- | |
| | | stearate | |
| | Emulgin B2 ® (Henkel) | Ceteareth-20 | 1.0 |
| | Lanette O ® (Henkel) | Cetyl and stearyl alcohol | 1.15 |
| | Myritol 318 ® (Henkel) | Caprylic/capronic acid | 5.0 |
| | | triglycerides | |
| | Cetiol SN ® (Henkel) | Cetyl and stearyl | 4.0 |
| | | isononanoate | |
| | Abil 100 ® | Polydimethylsiloxane | 1.0 |
| | (Goldschmidt) | | |
| | Bentone Gel MIO ® | Mineral oil and quaternium- | 3.0 |
| | (Rheox) | 18-hectorite and propylene | |
| | | carbonate | |
| | Cutina CBS ® (Henkel) | Glyceryl stearate and cetyl | 2.0 |
| | | alcohol and stearyl alcohol | |
| | | and cetyl palmitate and | |
| | | coconut glycerides | |
| | Neo Heliopan ® 303 | 2-Ethylhexyl 2-cyano-3,3- | 7.0 |
| | (Haarmann & Reimer) | diphenyl-2-propenoate | |
| | Neo Heliopan ® BB | 2-Hydroxy-4- | 1.0 |
| | (Haarmann & Reimer) | methoxybenzophenone | |
| | Neo Heliopan ® MA | Menthyl anthranilate | 3.0 |
| | (Haarmann & Reimer) | | |
| | 2-Ethylhexyl N,N- | | 3.0 |
| | dimethyl-4- | | |
| | aminobenzoate | | |
| | Titanium dioxide, | | 5.0 |
| | microfine | | |
| B | Water, dist. | | 55.65 |
| | Trilon BD ® (BASF) | Disodium EDTA | 0.1 |
| | Veegum ultra ® | Magnesium aluminum sulfate | 1.0 |
| | (Vanderbilt) | | |
| | Natrosol 250 HHR | Hydroxymethyl cellulose | 0.3 |
| | (Aqualon) | | |
| | Glycerol | | 3.0 |
| | Phenopip ® | 2-Phenoxyethanol and methyl | 0.3 |
| | (Nipa Laboratories) | 4-hydroxybenzoate and ethyl | |
| | | 4-hydroxybenzoate and propyl | |
| | | 4-hydroxybenzoate and butyl | |
| | | 4-hydroxybenzoate | |
| | 2-Hydrazino-1,3- | | 0.2 |
| | benzothiazole | | |
| C | Perfume oil | | 0.3 |

For part A, all the substances apart from the titanium dioxide were mixed and heated to 85° C.; the titanium dioxide was then carefully dispersed in the mixture. For part B, all the substances apart from Veegum and Natrosol were mixed and heated to 90° C.; the Natrosol and Veegum were then dispersed in the mixture, which was then added to part A while stirring. Part C was added to the mixture consisting of parts A and B and the mixture was then homogenized using a dispersing implement.

### Example 4

“Oil in water” sunscreen emulsion giving UVA/B broad-band protection

TABLE 4

| | | | Content |
|---|---|---|---|
| | Name of raw material | | in % by |
| Part | (manufacturer) | Chemical designation | weight |
| A | Crodaphos MCA ® | Cetyl phosphate | 1.50 |
| | (Croda) | | |
| | Cutina MD ® (Henkel) | Glyceryl stearate | 2.0 |
| | Lanette 16 ® (Henkel) | Cetyl alcohol | 1.2 |
| | Myritol 318 ® (Henkel) | Caprylic/capronic acid | 5.0 |
| | | triglycerides | |
| | Cetiol SN ® (Henkel) | Cetyl and stearyl | 5.0 |
| | | isononanoate | |
| | Copherol 1250 ® | Tocopherol acetate | 0.5 |
| | (Henkel) | | |
| | Solbrol P ® (Bayer) | Propyl 4-hydroxybenzoate | 0.1 |
| | Abil 100 ® | Polydimethylsiloxane | 0.3 |
| | (Goldschmidt) | | |
| | Trilon BD ® (BASF) | Disodium EDTA | 0.1 |
| | Neo Heliopan ® HMS | 3,3,5-Trimethylcyclohexyl | 5.0 |
| | (Haarmann & Reimer) | salicylate | |
| | Neo Heliopan ® 357 | Butylmethoxy- | 2.0 |
| | (Haarmann & Reimer) | dibenzoylmethane | |
| B | Water, dist. | | 47.6 |
| | 1,3-Butylene glycol | | 3.0 |
| | Sobrol M ® (Bayer) | Methyl 4-hydroxybenzoate | 0.2 |
| | Phenoxyethanol | | 0.7 |
| | Carbopol ETD 2050 ® | Acrylic acid/C₁₀-C₃₀-alkyl | 0.2 |
| | (B. F. Goodrich) | acrylate copolymer | |
| | Keltrol T ® (Calgon) | Xanthan gum | 0.2 |
| | Neo Heliopan ® AP | 2,2-(1,4-Phenylene)-bis-(1H- | 22 |
| | (Haarmann & Reimer) | benzimidazole-4,6-disulfonic | |
| | | acid) and disodium salt | |
| | 2-Hydrazino-1,3- | | 0.2 |
| | benzothiazole-5-sulfonic | | |
| | acid | | |
| | Aqueous sodium | | 2.8 |
| | hydroxide solution, 10% | | |
| D | Perfume oil | | 0.3 |
| | Bisabolol | | 0.1 |

Part A was heated to 850C. Carbopol and Keltrol were dispersed in the remaining constituents in the cold, after which the mixture was heated to 85° C. and added to part A. Part C was added immediately, at 80° C., to the mixture consisting of parts A and B and the whole was homogenized for 5 min using a dispersing implement. Finally, part D was added at room temperature and the mixture was homogenized using a dispersing implement.

### Example 5

Efficacy as free radical-capturing agents

The activity of the exemplary compounds as free radical-capturing agents was compared with that of conventional free radical-capturing agents. In this connection, use was made of the DPPH (1,1-diphenyl-2-picryl-hydrazyl) test for eliminating free radicals.

DPPH was dissolved in methanol to give a concentration of 100 μmol/l. A series of dilutions of the exemplary compounds, Vitamin C, α-tocopherol and dibutylhydroxytoluene were prepared in methanol. Methanol served as the control. 2500 μl of the DPPH solution were mixed with 500 μl of each test solution and the decrease in the absorption at 515 nm was read until the decrease was less than 2% per hour. The activity of the test substances as free radical-capturing agents was calculated using the following equation:

Activity as free radical-capturing agent (%)=100—(absorption of the test compounds)/(absorption of the control)×100.

For each test compound, the effective relative concentration EC₅₀ (based on the concentration of DPPH which was initially present, EC=c (test compound)/c(DPPH)) of a test compound, i.e. at which the DPPH free radical was 50% eliminated, was calculated from the activity as a free radical-capturing agent (%) in a series of dilutions of test compounds. The results are shown in Table 5:

TABLE 5

| | | EC₅₀/ |
|---|---|---|
| Test compound | CAS-No. | (mol/mol) |
| 2-Hydrazino-1,3-benzothiazole | 615-21-4 | 0.15 |
| 5,6-Dimethoxy-2-hydrazino-1,3- | — | 0.12 |
| benzothiazole | | |
| 2-Hydrazino-1,3-benzothiazole-5-sulfonic | 143269-94-7 | 0.17 |
| acid | | |
| 6-tert.-Butyl-2-hydrazino-1,3-benzothiazole | — | 0.15 |
| 6-Methyl-2-hydrazino-1,3-benzothiazole | 20174-69-0 | 0.28 |
| 2-Hydrazino-5-(4-fluorophenyl)-1,3,4- | — | 0.14 |
| thiadiazole | | |
| 2-Hydrazino-4-phenyl-1,3-thiazole | 34176-52-8 | 0.13 |
| 2-Hydrazino-thiazolo-[5,4-b]pyridine | 57135-11-2 | 0.10 |

Comparison:

| | |
|---|---|
| Vitamin C | 0.27 |
| α-Tocopherol | 0.25 |
| Dibutylhydroxytoluene | 0.24 |

### Example 6

Activity as antioxidants

The activity of the exemplary compounds as antioxidants was compared with that of conventional antioxidants. The accelerated autooxidation of lipids by air with or without antioxidant, as measured using the Rancimat appliance (Rancimat is a registered trademark belonging to Metrohm AG, Herisau, Switzerland) was used as the test system.

The exemplary compounds, Vitamin C, α-tocopherol and dibutyihydroxytoluene were dissolved in methanol or acetone, and 100 μl of the respective test solution were added to a previously prepared oil sample weighing 3 g. Solvent only was added to a control sample. A constant, dry stream of air (20 I/h) was blown through the heated oil sample containing the test solution, and the volatile oxidation products (principally short-chain fatty acids such as formic and acetic acid) were collected in a recipient vessel containing water. The conductivity of this aqueous solution was measured and documented continuously. In this connection, the oxidation of (unsaturated) fats proceeds only very slowly for a while and then suddenly increases rapidly. The time which elapses until the increase is termed the induction period (IP).

The antioxidative index (AOI) was obtained using the following equation:

AOI=IP_{(with test solution)}/IP_{(control sample)}.

The results for the experiments in soyabean oil at 100° C. and, at 80° C., in squalene, which was purified on alumina type N and stabilized with 1 ppm α-tocopherol, are shown in Table 6:

TABLE 6

| | AOl in Soyabean | AOl in squalene at |
|---|---|---|
| | oil at 100° C. | 80° C. containing |
| | containing 0.05% | 0.005% test |
| Test Compound | test substance | substance |
| 2-Hydrazino-1,3-benzothiazole | 17 | 97 |
| 5,6-Dimethoxy-2-hydrazino-1,3- | 4.1 | 24 |
| benzothiazole | | |
| 6-tert-Butyl-2-hydrazino-1,3- | 6 | 14 |
| benzothiazole | | |
| 6-Methoxy-2-hydrazino-1,3- | 24 | 23 |
| benzothiazole | | |
| 6-Methyl-2-hydrazino-1, | 2.4 | 67 |
| 3-benzothiazole | | |
| 2-Hydrazino-5-(4-fluorophenyl)- | 15 | 38 |
| 1,3,4-thiadiazole | | |
| 2-Hydrazino-4-phenyl-1, | 8.5 | 13 |
| 3-thiazole | | |
| 2-Hydrazino-thiazolo- | n.d. | 60 |
| [5,4-b]pyridine | | |

Comparison:

| | | |
|---|---|---|
| Vitamin C | 1.2 | 0.7 |
| α-Tocopherol | 5.1 | 39 |
| Dibutylhydroxytoluene | 4.8 | 38 |

n.d. = not determined

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

1. An antioxidant or free radical-capturing agent comprising 2-hydrazino-1,3-thiazoles of the general formula 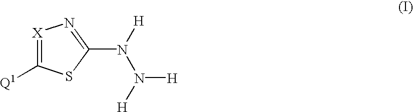
or their salts,
where
X represents a nitrogen atom or a substituted carbon atom C-Q², and either
Q¹ and Q² represent, independently of each other, hydrogen atoms, optionally hydroxyl- or alkyloxy-substituted unbranched, branched or cyclic alkyl, alkenyl, 1-oxoalkyl or 1-oxoalkenyl groups having from 1 to 18 carbon atoms, optionally substituted aryl groups having from 6 to 15 carbon atoms, optionally substituted heterocyclyl groups having from 2 to 15 carbon atoms and at least one heteroatom selected from the group oxygen, nitrogen and sulfur, optionally substituted arylalkyl or aryl-1-oxoalkyl groups having from 7 to 16 carbon atoms, halogen atoms, nitro groups or —COOR⁴, —OR⁴, —NR⁴R⁵, —SO₂OR⁴, —SO₂NR⁴R⁵ or —PO(OR⁴)(OR⁵) groups, or
Q¹ and Q² together represent a radical of the general formula (II) or
Q¹ and Q² together represent a radical of the general formula (II) 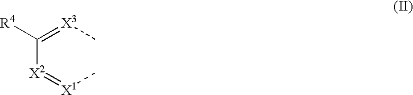
wherein
X¹, X² and X³ represent, independently of each other, either nitrogen atoms or carbon atoms having the radicals R¹, R² and R³, respectively, and
R¹, R² and R³ represent, independently of each other, hydrogen atoms, optionally hydroxyl- or alkyloxy-substituted unbranched, branched or cyclic alkyl, alkenyl, 1-oxoalkyl or 1-oxoalkenyl groups having from 1 to 18 carbon atoms, optionally substituted aryl groups having from 6 to 15 carbon atoms, optionally substituted heterocyclyl groups having from 2 to 15 carbon atoms and at least one heteroatom selected from the group oxygen, nitrogen and sulfur, optionally substituted arylalkyl or aryl-1-oxoalkyl groups of from 7 to 16 carbon atoms, halogen atoms, nitro groups or —COOR⁴, —OR⁴, —NR⁴R⁵, —SO₂OR⁴, —SO₂NR⁴R⁵ or —PO(OR⁴)(OR⁵) groups, and
R⁴ and R⁵ represent, independently of each other, hydrogen atoms, optionally hydroxyl- or alkyloxy-substituted unbranched, branched or cyclic alkyl, alkenyl, 1-oxoalkyl or 1-oxoalkenyl groups having from 1 to 18 carbon atoms, optionally substituted aryl groups having from 6 to 15 carbon atoms, or optionally substituted arylalkyl or aryl-1-oxoalkyl groups of from 7 to 16 carbon atoms.

2. An antioxidant or free radical-capturing agent comprising 2-hydrazino-1,3-thiazoles of the general formula (I) 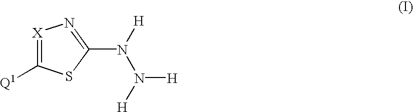
or their salts,
wherein
X represents a nitrogen atom or a Q²-substituted carbon atom C-Q², and either
Q¹ and Q² represent, independently of each other, hydrogen atoms, methyl, trifluoromethyl, ethyl, tert-butyl, allyl, 3-methylbut-2-en-1-yl or acetyl groups, chlorine or fluorine atoms, nitro groups, —COOR⁴, —OR⁴, —NR⁴R⁵, —SO₂OR⁴, —SO₂NR⁴R⁵ , —PO(OR⁴)(OR⁵) groups or phenyl, pyridyl, pyrazinyl, phenylmethyl or benzoyl groups which are optionally substituted on the aromatic moiety by alkyl, hydroxyl, alkyloxy, amino, dialkylamino, bromine, fluorine, chlorine, nitrile, sulfonic acid, sulfonamide or alkylsulfonate radicals, or
Q¹ and Q² together represent a radical of the general formula (II) 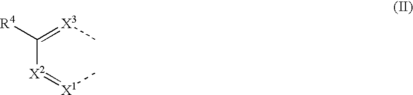
wherein
X¹, X² and X³ represent carbon atoms having the radicals R¹, R² and R³, respectively, and
R¹, R²and R³ represent, independently of each other, hydrogen atoms, methyl, trifluoromethyl, ethyl, tert-butyl, allyl, 3-methyl but-2-en-1-yl- or acetyl groups, phenyl, pyridyl, pyrazinyl, phenylmethyl, 4-methylphenylmethyl, 4-methoxyphenylmethyl or benzoyl groups, chlorine or fluorine atoms, nitro groups, or —COOR⁴, —OR⁴, —NR⁴R⁵, —SO₂OR⁴, —SO₂NR⁴R⁵ or —PO(OR⁴)(OR⁵) groups, and
R⁴ and R⁵ represent, independently of each other, hydrogen atoms, methyl, trifluoromethyl, ethyl, tert-butyl, allyl, 3-methylbut-2-en-1-yl, acetyl, propionyl or pivaloyl groups, or phenyl, pyridyl, pyrazinyl, phenylmethyl, 4-methylphenylmethyl, 4-methoxyphenylmethyl or benzoyl groups.

3. An antioxidant or free radical-capturing agent, wherein said antioxidant or free radical-capturing agent is 2-hydrazino-1,3-benzothiazole, 5,6-dimethoxy-2-hydrazino-1,3-benzothiazole, 6-methoxy-2-hydrazino-1,3-benzothiazole, 2-hydrazino-1,3-benzothiazole-5-sulfonic acid, 2-hydrazino-1,3-benzothiazole-6-sulfonic acid, 6-tert-butyl -2-hydrazino-1,3-benzothiazole, 6-methyl-2-hydrazino-1,3-benzothiazole, 2-hydrazino-thiazolo-[5,4-b]pyridine, 2-hydrazino-5-(4-fluorophenyl) -1,3,4-thiadiazole, 2-hydrazino-4-phenyl-1,3-thiazole and 2-hydrazino -4-methyl-1,3-thiazole hydrochloride.

4. A cosmetic and dermatological preparations, comprising 2-hydrazino-1,3-thiazoles of the general formula (I) 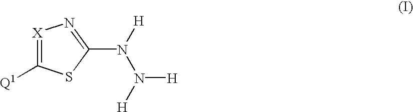
or their salts,
where
X represents a nitrogen atom or a Q²-substituted carbon atom C-Q², and
either
Q¹ and Q² represent, independently of each other, hydrogen atoms, methyl, trifluoromethyl, ethyl, tert-butyl, allyl, 3-methylbut-2-en-1-yl or acetyl groups, chlorine or fluorine atoms nitro groups, —COOR⁴, —OR⁴, —NR⁴R⁵, —SO₂OR⁴, —SO₂NR⁴R⁵ or —PO(OR⁴)(OR⁵) groups or phenyl, pyridyl, pyrazinyl, phenylmethyl or benzoyl groups which are optionally substituted on the aromatic moiety by alkyl, hydroxyl, alkyloxy, amino, dialkylamino, bromine, fluorine, chlorine, nitrile, sulfonic acid, sulfonamide or alkylsulfonate radicals, or
Q¹ and Q² together represent a radical of the general formula (II) 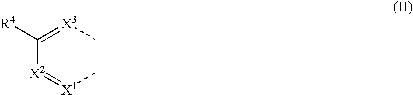
wherein
X¹, X² and X³ represent carbon atoms having the radicals R¹, R² and R³, respectively, and
R¹, R² and R³ represent, independently of each other, hydrogen atoms, methyl, trifluoromethyl, ethyl, tert-butyl, allyl, 3-methylbut-2-en-1-yl- or acetyl groups, phenyl, pyridyl, pyrazinyl, phenylmethyl, 4-methylphenylmethyl, 4-methoxyphenylmethyl or benzoyl groups, chlorine or fluorine atoms, nitro groups, or —COOR⁴, —OR⁴, —NR⁴R⁵, —SO₂OR⁴, —SO₂NR⁴R⁵ or —PO(OR⁴)(OR⁵) groups, and
R⁴ and R⁵ represent, independently of each other, hydrogen atoms, methyl, trifluoromethyl, ethyl, tert-butyl, allyl, 3-methylbut-2-en-1-yl, acetyl, propionyl or pivaloyl groups, or phenyl, pyridyl, pyrazinyl, phenylmethyl, 4-methylphenylmethyl, 4-methoxyphenylmethyl or benzoyl groups.

5. Cosmetic and dermatological preparations according to claim 4, which comprise from 0.0001% by weight to 30% by weight of the 2-hydrazino-1,3-thiazoles based on the total weight of the preparations.

6. Cosmetic and dermatological preparations according to claim 5, which comprise from 0.001 to 20% by weight of the 2-hydrazino-1,3-thiazoles based on the total weight of the preparations.

7. Cosmetic and dermatological preparations according to claim 6, which comprise from 0.01 to 5% by weight of the 2-hydrazino-1,3-thiazoles based on the total weight of the preparations.

8. Preparations which are of use in nutrition or consumption comprising 2-hydrazino-1,3-thiazoles of the general formula (I) 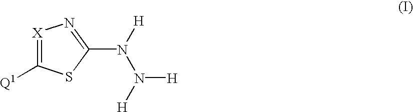
or their salts,
where
X represents a nitrogen atom or a Q²-substituted carbon atom C-Q², and either
Q¹ and Q² represent, independently of each other, hydrogen atoms, methyl, trifluoromethyl, ethyl, tert-butyl, allyl, 3-methylbut-2-en-1-yl or acetyl groups, chlorine or fluorine atoms nitro groups, —COOR⁴, —OR⁴, —NR⁴R⁵, —SO₂OR⁴, —SO₂NR⁴R⁵ or —PO(OR⁴)(OR⁵) groups or phenyl, pyridyl, pyrazinyl, phenylmethyl or benzoyl groups which are optionally substituted on the aromatic moiety by alkyl, hydroxyl, alkyloxy, amino, dialkylamino, bromine, fluorine, chlorine, nitrile, sulfonic acid, sulfonamide or alkylsulfonate radicals, or
Q¹ and Q² together represent a radical of the general formula (II) 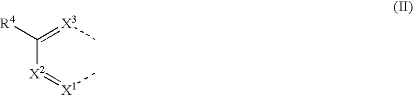
wherein
X¹, X² and X³ represent carbon atoms having the radicals R¹, R² and R³, respectively, and
R¹, R² and R³ represent, independently of each other, hydrogen atoms, methyl, trifluoromethyl, ethyl, tert-butyl, allyl, 3-methylbut-2-en-1-yl- or acetyl groups, phenyl, pyridyl, pyrazinyl, phenylmethyl, 4-methylphenylmethyl, 4-methoxyphenylmethyl or benzoyl groups, chlorine or fluorine atoms, nitro groups, or —COOR⁴, —OR⁴, —NR⁴R⁵, —SO₂OR⁴, —SO₂NR⁴R⁵ or —PO(OR⁴)(OR⁵) groups, and
R⁴ and R⁵ represent, independently of each other, hydrogen atoms, methyl, trifluoromethyl, ethyl, tert-butyl, allyl, 3-methylbut-2-en-1-yl, acetyl, propionyl or pivaloyl groups, or phenyl, pyridyl, pyrazinyl, phenylmethyl, 4-methylphenylmethyl, 4-methoxyphenylmethyl or benzoyl groups.

9. Preparations which are of use in nutrition or consumption according to claim 8, which comprise from 0.0001% by weight to 30% by weight of the 2-hydrazino-1,3-thiazoles based on the total weight of the preparations.

10. Preparations which are of use in nutrition or consumption according to claim 9, which comprise from 0.001 to 20% by weight of the 2-hydrazino-1,3-thiazoles based on the total weight of the preparations.

11. Preparations which are of use in nutrition or consumption according to claim 10, which comprise from 0.01 to 5% by weight of the 2-hydrazino-1,3-thiazoles based on the total weight of the preparations.

12. Preparations according to claim 4 which additionally comprise at least one UVA- and/or UVB-filtering substance.

13. Preparations according to claim 4, which additionally comprise at least one further antioxidant or free radical-capturing agent.

14. Preparations according to claim 4, which additionally comprise at least one UVA- and/or UVB-filtering substance and at least one further antioxidant or free radical-capturing agent.
